(19) United States
(12) Patent Application Publication
(10) Pub. No.: US 2014/0012194 A1
(43) Pub. Date: Jan. 9, 2014

(54) THREE WAY VALVE FOR MEDICAL DEVICE

(71) Applicant: Cook Medical Technologies LLC, Bloomington, IN (US)

(72) Inventors: Matthew J. Terwiske, Bloomington, IN (US); Justin D. Renfrow, Bloomington, IL (US); Neal Bridgett, Bloomington, IN (US)

(21) Appl. No.: 13/932,389

(22) Filed: Jul. 1, 2013

Related U.S. Application Data
(60) Provisional application No. 61/667,663, filed on Jul. 3, 2012.

(51) Int. Cl. A61M 25/00; A61M 25/10

(52) U.S. Cl. CPC A61M 25/0097; A61M 25/10
USPC 604/99.04; 604/246

(57) ABSTRACT

A device for performing a medical procedure is provided. The device includes an elongate catheter disposed between a distal end portion and a proximal end portion, with a central portion therebetween. A central portion of the catheter includes parallel first and second lumens therethrough, wherein the first lumen extends through in the distal end portion and the second lumen extends through the distal end portion. A fluid input connection is provided along with a selection valve stem disposed within a valve housing.

### CROSS-REFERENCE TO RELATED APPLICATIONS

This application claims priority from U.S. Provisional Application No. 61/667,663, filed on Jul. 3, 2012, the entirety of which is fully incorporated by reference herein.

### TECHNICAL FIELD

Hysterography, also known as mapping of the uterus, is a procedure used to examine the inside of the uterus. It is performed by threading a catheter transcervically into the uterus. The uterus is sealed and an image enhancing medium is injected into the uterus to allow the inside of the uterus to be viewed and diagnosed by using a fluoroscope, ultrasound, x-ray, or other device or technique. Sonohysterography (SHG) is a procedure involved with mapping or otherwise obtaining images of the uterus with the use of ultrasound. Hysterosalpingography (HSG) is a procedure to investigate the shape and patency of the uterus and fallopian tubes, which is performed with a contrast media under radiography.

### BRIEF SUMMARY

A first representative embodiment of the disclosure is provided. The embodiment is a device for performing a medical procedure. The device includes an elongate catheter disposed between a distal end portion and a proximal end portion, with a central portion therebetween. The catheter includes a central portion with parallel first and second lumens therethrough, wherein the first lumen fluidically communicates with an aperture within the distal end portion and the second lumen fluidically communicates with an inflatable balloon disposed upon the catheter. A fluid input connection is provided upon a housing and a selection valve stem is disposed within the housing. The valve stem is rotatable mounted within a housing to allow selective fluid communication between the fluid input and either the first or second lumen.

A second representative embodiment of the disclosure is provided. The embodiment is a method of performing a medical procedure. The method includes the steps of inserting an elongate catheter configured for completion of a medical procedure into a patient, wherein the catheter comprises an aperture disposed upon a distal end portion of the catheter and an inflatable balloon disposed upon the catheter. The aperture and balloon are each configured to selectively and independently receive fluid from a single fluid input disposed upon the housing through a central portion. The catheter is fixed to a housing that pivotably supports a valve stem, wherein the position of the valve stem with respect to the housing allows selective fluid communication from the input source to one of the aperture or balloon. The valve stem is aligned for fluid communication from the input source to the aperture, and the aligning the valve stem for fluid communication from the input source to the balloon.

Advantages of the present disclosure will become more apparent to those skilled in the art from the following description of the preferred embodiments of the disclosure that have been shown and described by way of illustration. As will be realized, the disclosed subject matter is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a perspective view of a catheter with a selective porting mechanism.

FIG. 2 is a cross-sectional view of the catheter of FIG. 1 with the selection mechanism configured to port fluid to an outlet aperture.

FIG. 3 is the view of FIG. 2 with the selection mechanism configured to port fluid to an inflatable balloon.

FIG. 4 is a perspective view of the catheter with the housing removed, showing the selection mechanism configured to port fluid to the outlet aperture.

FIG. 5 is the view of FIG. 4 with the selection mechanism configured to port fluid to the balloon.

FIG. 6 is a partial cross-sectional view of the catheter of FIG. 1, with the selection mechanism in an intermediate position.

FIG. 7 is the view of FIG. 4 with the selection mechanism configured to block flow through the catheter.

FIG. 8 is a perspective view of a second catheter with a selective porting mechanism.

### DETAILED DESCRIPTION OF THE DRAWINGS AND THE PRESENTLY PREFERRED EMBODIMENTS

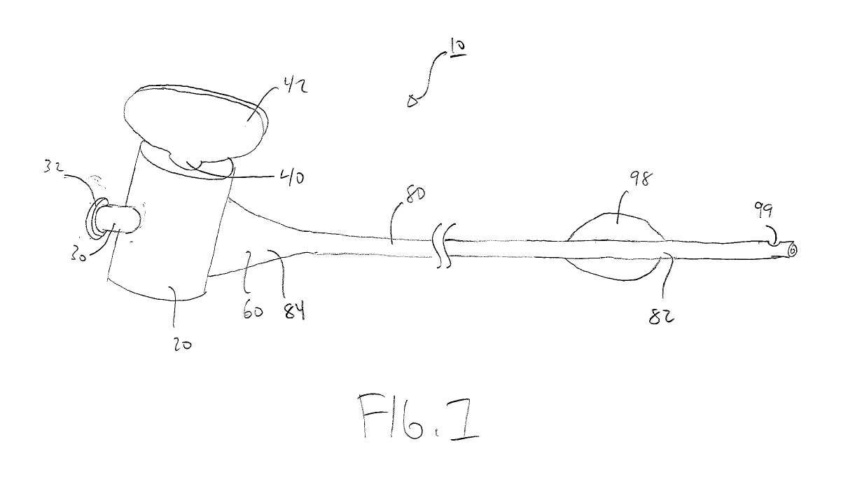

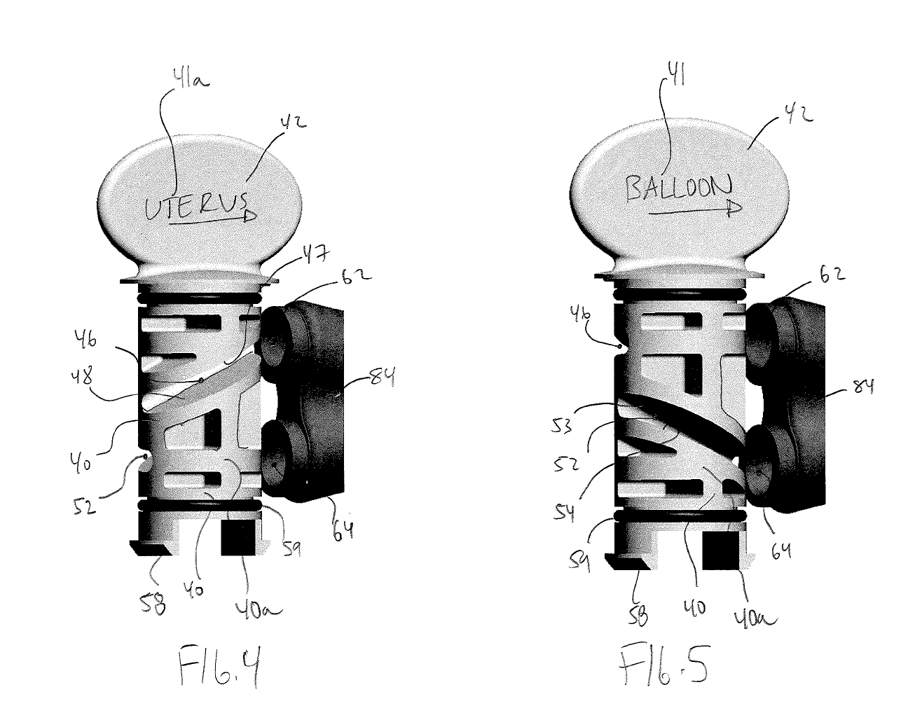

Turning now to FIGS. 1-7, a catheter 10 is provided that is configured for assistance with a medical procedure, such as an SHG or HSG procedure. The catheter 10 extends between a distal end portion 82 and a proximal end portion 84, with a central portion 85 disposed therebetween. The distal end portion 82 may include an aperture 99 that is disposed upon the tip of the distal end portion 82, or in other embodiments, proximal of the tip and disposed upon a circumferential surface of the distal end portion 82. The distal end portion 82 may further include a balloon 98 or other expandable member, such as a Foley balloon. The balloon 98 may be disposed proximal of the aperture 99. As discussed in greater detail below, both of the aperture 99 and the balloon 98 may be selectively and independent fluidically connected to an input and source of fluid, such as through an input connection 30, which may receive a syringe or other apparatus that is matably received upon the input connection 30 with a Luer lock connection 32 or the like.

The central portion 85 of the catheter 10 may be formed with parallel and independent first and second lumens 86, 88, which provide fluid communication from an input housing 20 and ultimately to the aperture 99 and the balloon 98. Specifically, the central portion 85 is configured such that the first lumen 86 extends therethrough and is fluidly connected to the aperture 99, and the second lumen 88 extends through the central portion, in parallel with the first lumen 86, and is fluidly connected to the balloon 98, such that fluid flowing through the second lumen 88 (at the pressure supplied through the input connection 30) inflates the balloon 98, and a release of pressure allows the balloon 98 to deflate. The first and second lumens 86, 88 may be separated by a central wall such that the first and second lumens 86, 88 define opposite portions of the cross-section of the central portion 85, while in other embodiments, the central portion 85 may be formed such that the first and second lumens 86, 88 are formed coaxially, for example with the first lumen 86 partially or fully enclosed by the second lumen 88, with a wall separating the two lumens 86, 88 along the length of the central portion 85.

The proximal end portion 84 of the catheter 80 is configured to mate with the housing 20, and specifically includes an output hub 60 that includes first and second inputs 62, 64 that each mate with respective first and second output holes 44, 45 in the housing 20. The first input 62 in the catheter 80 is in fluid communication with the first lumen 86 (and ultimately the aperture 99 disposed upon the distal end portion 82 of the catheter 10) and the second input 64 is in fluid communication with the second lumen 88 (and ultimately the balloon 98). In some embodiments, the output hub 60 has a decreasing height as it extends away from the housing 20 to transition to a size similar to the catheter 80.

The housing 20 may either be fixably mounted to the proximal portion 84 of the catheter 80 (either with the output hub 60, or through another attachment structure), or may be monolithically or integrally formed with the proximal portion 84. In some embodiments the housing 20 and the output hub 60 may be fixed together, or constructed monolithically or integrally.

The housing 20 receives a valve stem 40 rotatably disposed therein, which is rotatably mounted within the housing 20 to allow selective fluid communication between a fluid input 30 and one of the first or second inputs 62, 64 of the catheter 80. The housing 20 may be substantially cylindrical or in other shape or geometry, and may be configured to interact with the valve stem 40 to properly direct fluid from the single input 30 to the desired portion of the catheter 80 in a single handed operation, such as by rotating an operator 42 disposed upon the valve stem 40 and extending from the housing 20.

The valve stem 40 may be configured with two independent indentations 46, 52 that provide selective fluid paths between the single input 30 and one of the first or second inputs 62, 64 within the output hub 60 (through the respective outputs 44, 45 of the housing 20), depending upon the orientation of the operator 42. The indentations 46, 52 are each configured to prevent flow both when the stem 40 is aligned for flow through the opposite indentation 46, 52 and also when the valve stem 40 is aligned in an intermediate position, which in some embodiments is an alignment of the valve stem 40 with the operator 42 perpendicular to the direction of flow through the housing 20.

Each indentation 46, 52 may be disposed on opposite circumferential sizes of the valve stem, and each indentation may be disposed upon the valve stem such that they each form a helical profile upon the outer circumference of the valve stem 40. In some embodiments, the indentations 46, 52 may be disposed substantially opposite to each other, such that an entrance 46a to the first indentation 46 is disposed on an opposite portion of the valve stem 40 with an entrance 52a of the second indentation 52, and vice versa, such that an exit 46b of the first valve stem 46 is disposed on an opposite portion of the valve stem 40 with an exit 52b of the second indentation 52. Similarly, the midpoint of each indentation 46, 52 may be disposed opposite from each other.

In some embodiments, the entrance 46a, 52a to each of the first and second indentations 46, 52 may be disposed at the same vertical position upon the valve stem 40, in order to communicate with the single input 30 when so-positioned. The first and second indentations 46, 52 may be disposed in opposite orientations, such that the first indentation 46 traverses a generally upward direction along the valve stem 40 to interact with the first input 64, and the second indentation 52 traverses a generally downward direction along the valve stem 40 to interact with the second input 66.

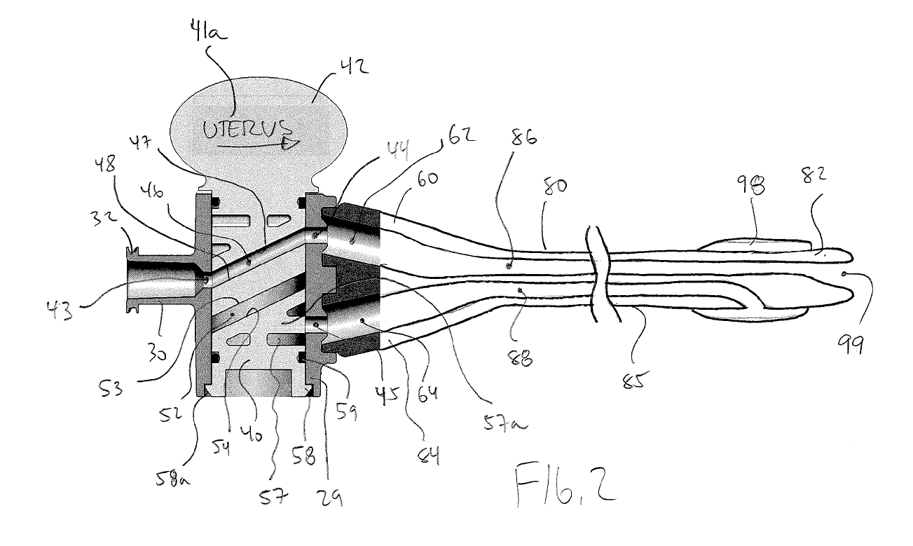

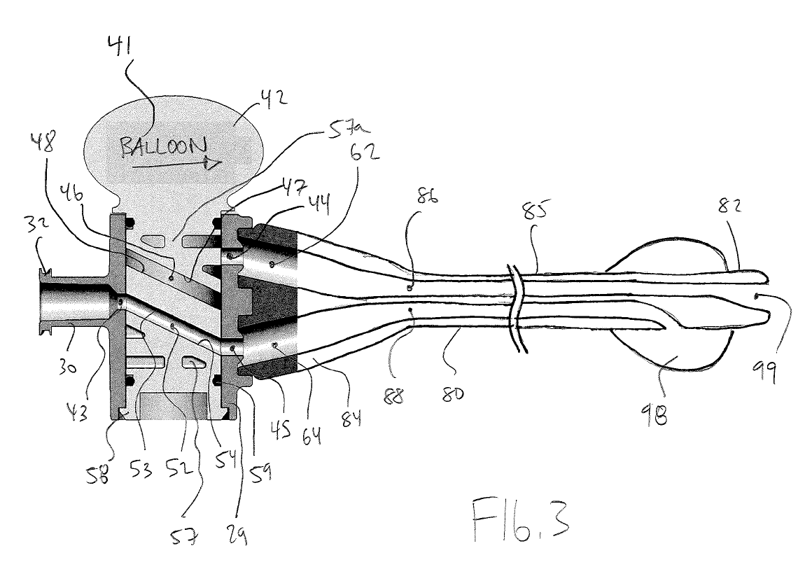

The first and second indentations 46, 52 may each be defined by respective upper and lower flat or planar surfaces 47, 48 and 53, 54. In some embodiments and as best shown in FIGS. 2 and 3, one or both of the indentations 46, 52 may form a straight path through the valve stem 40, with the circumferential edges of the indentations forming parallel helixes upon the outer circumference of the valve stem 40. As best shown in FIGS. 2 and 3, in some embodiments, the first and second indentations 46, 52 may each extend through at least just more than half of the diameter of the valve stem 40 (at the deepest point of each indentation), such that a vertical planar sectional cut through the central axis of the valve stem (as shown in FIGS. 2 and 3) shows both the first and second indentations 46, 52. This size and shape of each indentation maximizes the size of the flowpath through each of the first and second indentations 46, 52 and therefore minimize any headloss therethrough, when the valve stem 40 is oriented to port fluid form the single input 30 through the catheter 80 to one of the balloon 98 or the aperture 99. The valve stem 40 may include a plurality of cutouts 57 to minimize the amount of material used to construct the valve stem 40 with a plurality of stiffening ribs 57a to provide sufficient strength to the valve stem 40. In some embodiments, the inner wall of one or both indentations may be arcuate, while in other embodiments the inner wall may be flat, or angled.

The valve stem 40 may be rotatably mounted within the housing 20 such that it is able to rotate a full 360 degrees thereabout, while in other embodiment, the valve stem 40 may be constructed such that it may be rotated only 180 degrees about the housing (with the intermediate position at the center of the allowed rotation and the orientations providing registry with the first and second inputs 62, 64 of the catheter 80 at opposite bounds of the range of motion. In some embodiments, the valve stem 40 is constructed with two or more feet 58 that mate with a ledge 29 in the housing 20 to retain the valve stem 40 within the housing 20, with the feet 58 being inwardly (and in some embodiments, include lower ramp surfaces 58a to allow the feet 58 to move inwardly to allow the valve stem 40 to be initially positioned within the housing 20. The valve stem 40 may mate with the housing 20 with two or more o-rings that are disposed within slots in the valve stem 40 to prevent leakage of fluid from the valve stem 40 out of the housing 20.

The valve stem 40 may include an operator 42 that extends from an upper (or lower) surface of the housing 20 and allows for easy and single handed rotation of the valve stem 40 to selectively port fluid from the single input 30 and ultimately to the balloon 98 or the aperture 99, by way of the catheter 80. In some embodiments, the operator 42 may be disposed in parallel to a plane between the fluid input 30 and the first and second outputs 62, 64 when the valve stem 40 is positioned to port fluid from the single input 30 to either of the first or second outputs 62, 64. In some embodiments, the operator 42 may be positioned perpendicular to the plane between the fluid input 30 and the first and second outputs 62, 64 when the valve stem 40 is positioned to prevent fluid flow therethrough, which provides the operator with a quick and convenient visual and/or tactile indication of the valve position. In some embodiments, the operator 42 may have a verbal or pictorial indication 41a of valve position, such as the word “Uterus” or the like with a corresponding arrow or other marker pointing to the first and second outlets 62, 64 when the valve stem 40 is positioned to port fluid to the aperture 99, and the word “Balloon” with a corresponding arrow or other marker 41, which points to the first and second outlets 62, 64 when the valve stem 40 is ported to balloon 98.

In other embodiments, the housing 20 may include one, two, or more windows that provide a view of the valve stem 40 therewithin, which provides the operator with an indication of the valve position 40, and specifically how the valve stem 40 is oriented to port fluid therethrough. In some embodiments, the window(s) may be provided in conjunction with indications disposed upon the housing 20 to provide visual clarification about the position of the valve stem 40 and the porting of fluid through the housing 20.

The fluid input connection 30 is provided to receive a supply of pressurized fluid therethrough, such as through a syringe. The fluid input 30 may include a Luer lock fitting, or other conventional fitting or attachment device, to allow for a fluid seal with a syringe.

In other embodiments, shown in FIG. 8, catheter 100 is provided. The catheter 100 may be configured in a similar manner to catheter 10 discussed above, and for the sake of brevity, similar structure with catheter 100 is referenced with reference numbers used to describe catheter 10, above. Catheter 100 may include a modified distal end portion 182 and a proximal end portion 84 with a central portion therebetween. The proximal end portion 84 may extend from an output hub 60 that extends from a housing 20 with a valve stem 40 that selectively ports fluid flowing from a single input 30 to one of the first or second inputs 62, 64 of the output hub 60. The distal end portion 182 includes first and second lumens 86, 88 that are fluidically connected to the housing 20 by way of the output hub 60, such that the first lumen 86 is fluidly connected to the first input 62, and the second lumen 88 is fluidly connected to the second input 64. Similar to the device 10, the valve stem 40 is configured with two independent indentations 46, 52 to provide selective flow paths between the single input 30 and the first and second inputs 62, 64 of the hub, based upon the rotational alignment of the valve stem 40 with respect to the housing 20.

The distal end portion 182 of the catheter 100 may include first and second balloons 192, 194 that are each fluidly connected to the respective first and second lumens 86, 88 of the distal end portion 182, such that fluid flowing through the first lumen 86 flows to inflate (or deflate, depending upon the direction of flow) the first balloon 192 and fluid flowing through the second lumen 88 flows to inflate (or deflate, depending upon the direction of flow) the second balloon 194. As can be understood with reference to FIG. 8 (and understanding of the structure and operation of the housing 20 and the valve stem 40), the first and second balloons 192, 194 may be selectively inflated (and maintained in an inflated configuration) and selectively deflated based upon the position of the valve stem 40 with respect to the housing 20 and the existence of a source of pressure attached to the single input 30. In other embodiments, the distal end portion 182 could be configured with two apertures (instead of two balloons) that are each fluidly connected to one of the first and second lumens 86, 88 to allow for selective flow through the lumens 86, 88 and out of the apertures based upon the existence of pressure at the single input 30 and the position of the valve stem 40 with respect to the housing 20.

In some embodiments, a method of performing a medical procedure is provided which uses the device 10. The method may involve inserting the catheter 80 of the device into the patient, such as into the patient's vagina and through the patient's cervix and into the uterus, which may be so implanted by a physician or other medical professional using common knowledge and clinical experience. The proximal end portion 84 of the catheter 80, the output hub 60, the housing 20, and the input 30 of the device extend out of the patient and may be manipulated by the physician to both guide the distal end portion 80 into the proper position as well as to manipulate the valve stem 40 and fluid source, as discussed below. The method may include or assist with the performance of a SHG or HSG procedure, or a similar medical procedure in a patient (human or mammal).

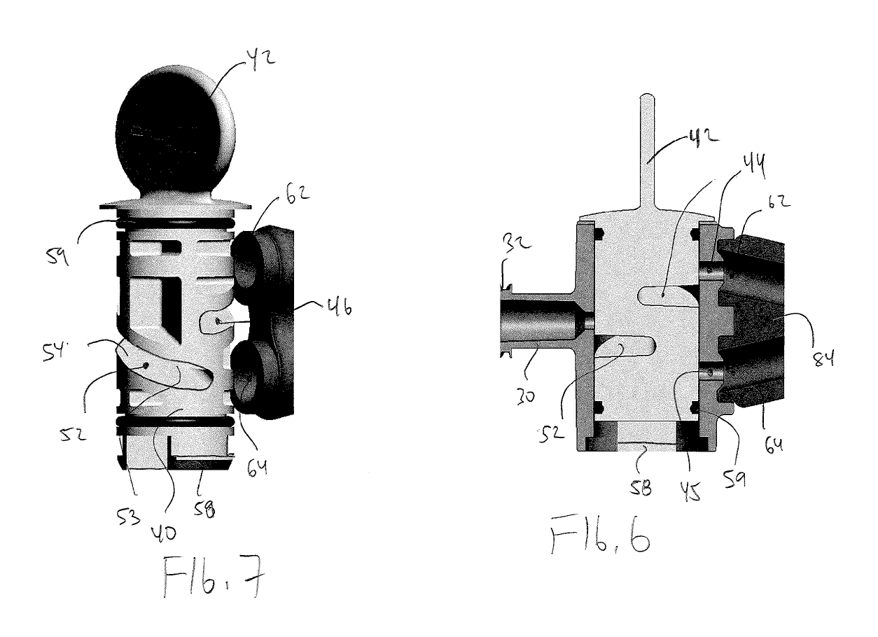

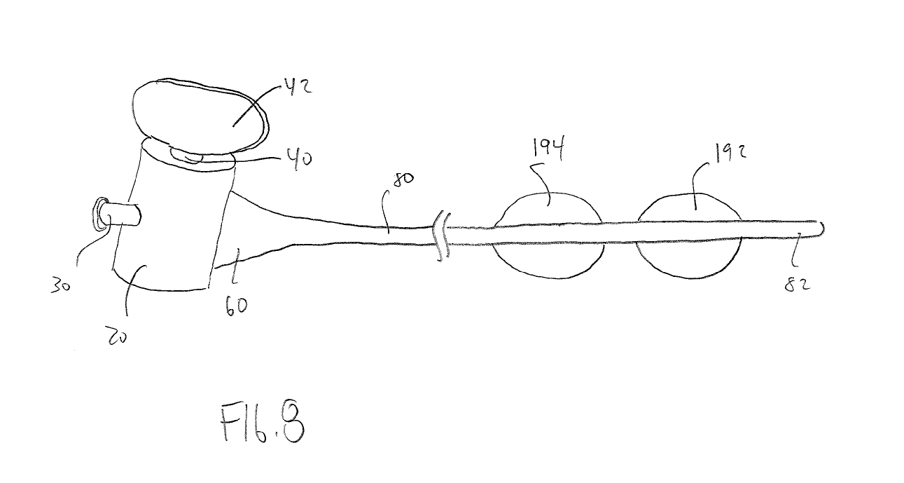

Once the device 10 is properly positioned, the physician may pivot the valve stem 40 to the “Balloon” position by turning the operator 42 such that the arrow associated with the indication 41 (such as the word “Balloon”) is pointing toward the output hub 60, (or by confirming that the valve stem 40 is currently in this position). The physician then may fluidly connect the fluid input 30 with a syringe and inject the rated volume of fluid into the catheter to inflate the balloon 98. Once the balloon has been filed, which may be ascertained by observing the amount of fluid injected from the syringe, the physician rotates the operator to the intermediate position (FIGS. 6 and 7), which prevents backflow from the balloon 98 through the catheter 80 and therefore maintains the balloon in the inflated configuration.

The physician then rotates the operator 42 to the “Uterus” position by turning the operator 42 such that the arrow associated with the indication 41a (such as the word “Uterus”) is pointing toward the output hub 60. The physician then injects additional fluid into the input 30 as needed for the procedure, which extends through the catheter 80 and out the aperture 99 and into the uterus (assuming that the balloon 98 of the device 10 is maintained with traction against the cervix). If more fluid than can be provided by one syringe is needed, the physician may temporarily port the valve stem 40 to the intermediate position, to prevent backflow through the catheter 80, and hook up a second syringe (or other fluid input source) and then return the operator 42 to the “Uterus” position (or other position to port fluid to the aperture 99). At the conclusion of the position, the physician may port a source of suction to the input 30 (with the operator 42 in the “Uterus” position) to remove excess fluid from the uterus through the catheter 80. The physician then may rotate the operator 42 to align the valve stem 40 in the “Balloon” position to allow fluid to flow from the balloon 98, either as urged by a source of suction disposed upon the inlet 30, or due to the pressure of the fluid disposed in the balloon 98 if the input 30 is open to the atmosphere. The catheter 80 may then be removed from the patient.

In other embodiments, a method of performing a medical procedure is provided with uses the device 100, such as a cervical ripening procedure to assist with labor. In use, the device 100, which includes first and second balloons 192, 194 may be positioned within the patient such that the distal-most first balloon 192 is within the uterus and the proximal-most second balloon 194 is within the patient's vagina with the cervix therebetween. A source of fluid, such as a syringe, is connected to the single input 30 through a luer lock connector (or other suitable connection structure) and the valve stem 40 is rotated to a position to port fluid from the single input 30 through the first input 62 to the hub 60, the first lumen 86 and into the first balloon 192 (when fluid is injected into the single input 30 from the syringe), by observing (or tactily feeling) the position of the operator 42 (and the indication 41a thereon) with respect to the housing 20. The physician (or other medical professional) may observe the amount of fluid ultimately ported to the first balloon 192 (such as by observing the fluid leaving the syringe) and when the correct volume of fluid has been ported to the first balloon 192, the physician may rotate the valve stem 40 to the intermediate position (FIGS. 6, 7) to hold the fluid within the first balloon 192.

The physician may then provide proximal traction on the catheter 100 and rotate the valve stem 40 to port to the second balloon 194) as observed (or tactily felt) based upon the position of the operator 42 (or the indication 41a) with respect to the housing 20. The physician may then apply fluid to the single input 30, through the second indentation 52, the second input 62 to the hub 60, the second lumen 88 and ultimately to the second balloon 194, which is properly inflated, potentially by observing the amount of fluid ported to the device 100 through the syringe. The physician may then selectively continue to inflate the first and/or second balloons 192, 194 as needed for the clinical cervical ripening procedure, by selectively rotating the valve stem 40 while observing the operator position (or tactily feeling) to ensure that the fluid is ported correctly. After the procedure is stopped (potentially, when the cervix is fully ripened), the fluid pressure is removed from each balloon by porting the operator 42 of the valve stem 40 to the desired first or second balloon 192, 194, which allows the fluid to flow through the respective first or second balloon 192, 194 and ultimately out of the inlet 30, based upon the position of the valve stem 40.

While the preferred embodiments of the disclosure have been described, it should be understood that the disclosure is not so limited and modifications may be made without departing from the disclosure. The scope of the invention is defined by the appended claims, and all devices that come within the meaning of the claims, either literally or by equivalence, are intended to be embraced therein.

1. A device for performing a medical procedure, comprising:
an elongate catheter disposed between a distal end portion and a proximal end portion, with a central portion therebetween;
the central portion defining parallel first and second lumens therethrough;
a fluid input connection; and
a selection valve stem disposed within a valve housing, wherein the valve stem is rotatably mounted within a housing to allow selective fluid communication between the fluid input and either the first or second lumen.

2. The device of claim 1, wherein the first lumen fluidically communicates with an aperture within the distal end portion and the second lumen fluidically communicates with an inflatable balloon disposed upon the catheter.

3. The device of claim 1, wherein one or both of the first and second lumens fluidically communicates with a respective inflatable balloon.

4. The device of claim 1, wherein the valve stem is cylindrical and comprises a plurality of indentations that traverse along different portions of the valve stem.

5. The device of claim 4, wherein the plurality of indentations comprise a first indentation disposed in a generally downward orientation and a second indentation extending in a generally upward direction and disposed on an opposite circumferential side of the valve stem from the first indentation.

6. The device of claim 5, wherein the first and second indentations each are defined by respective upper and lower planar surfaces.

7. The device of claim 5, wherein the first and second indentations each define a helical slot upon an outer circumferential surface of the valve stem.

8. The device of claim 5, wherein the first and second indentations are each configured to prevent fluid communication between the fluid input connection and the first and second lumens when the valve stem is in an intermediate position.

9. The device of claim 8, wherein the valve stem further comprises an operator, wherein the operator is disposed substantially perpendicular to a plane through the fluid input connection when the valve stem is in the intermediate position, and parallel to the plane when one of the first or second indentations is in registry with the fluid input connection.

10. The device of claim 5, wherein the housing comprises a window and the valve stem comprises a plurality of visual indicators that when in registry with the window display an operational configuration of the device.

11. The device of claim 5, further comprising an exit conduit rigidly connected to the valve housing, the exit conduit comprising a first path in fluid communication with the first lumen and configured to selectively receive fluid through the first indentation, and a second path in fluid communication with the second lumen and configured to selectively receive fluid through the second indentation.

12. The device of claim 11, wherein a height of the exit conduit decreases as its distance from the housing increases.

13. A method of performing a medical procedure, comprising:
inserting an elongate catheter configured for completion of a medical procedure into a patient, wherein the catheter comprises an aperture disposed upon a distal end portion of the catheter and an inflatable balloon disposed upon the catheter, wherein the aperture and balloon are each configured to selectively and independently receive fluid from a single fluid input disposed upon the housing through a central portion, the catheter being fixed to a housing that pivotably supports a valve stem, wherein the position of the valve stem with respect to the housing allows selective fluid communication from the input source to one of the aperture or balloon;
aligning the valve stem for fluid communication from the input source to the aperture; and
aligning the valve stem for fluid communication from the input source to the balloon.

14. The method of claim 13, further comprising a step of aligning the valve stem to prevent fluid communication from the input source to the central portion of the catheter and both of the first and second distal ends.

15. The method of claim 13, further comprising a step of rotating an operator of the valve stem to alter the alignment of the valve stem, wherein the operator comprises a visually perceptible indicator to communicate the orientation of the valve stem with respect to the fluid input.

16. The method of claim 13, wherein the catheter further comprises a first lumen disposed between the housing and the aperture and a parallel second lumen disposed between the housing and the balloon.

17. The method of claim 13, wherein the medical procedure is one of a sonohysterography or a hysterosalpingography procedure.

18. A method of performing a medical procedure, comprising:
inserting an elongate catheter configured for completion of a medical procedure into a patient, wherein the catheter comprises a first inflatable balloon disposed upon a distal end portion of the catheter and second inflatable balloon disposed upon the catheter proximally of the first balloon, wherein the first and second inflatable balloons are each configured to selectively and independently receive fluid from a single fluid input disposed upon the housing through a central portion, the catheter being fixed to a housing that pivotably supports a valve stem, wherein the position of the valve stem with respect to the housing allows selective fluid communication from the input source to one of the aperture or balloon;
aligning the valve stem for fluid communication from the input source to the first balloon; and
aligning the valve stem for fluid communication from the input source to the second balloon.

19. The method of claim 18, wherein the medical procedure is a cervical ripening procedure.
